(19) United States
(12) Patent Application Publication
(10) Pub. No.: US 2025/0078513 A1
(43) Pub. Date: Mar. 6, 2025

(54) Online Exam Proctoring System

(71) Applicant: Fusemachines Inc., New York, NY (US)

(72) Inventors: Prajin KHADKA, Kathmandu (NP); Rakesh KUMAR KATUWAL, Kathmandu (NP); Anish JOSHI, New York, NY (US); Sameer MASKEY, New York, NY (US)

(21) Appl. No.: 18/599,110

(22) Filed: Mar. 7, 2024

Related U.S. Application Data
(60) Provisional application No. 63/450,530, filed on Mar. 7, 2023.

(51) Int. Cl. G06V 20/52; A61B 5/1171; A61B 5/16; G06T 7/70; G06V 10/82; G06V 20/40; G06V 40/16; G09B 5/08; G10L 17/06; G10L 17/18; G10L 25/57

(52) U.S. Cl. CPC G06V 20/52; A61B 5/1176; A61B 5/163; A61B 5/164; G06T 7/70; G06V 10/82; G06V 20/40; G06V 40/161; G06V 40/172; G09B 5/08; G10L 17/06; G10L 17/18; G10L 25/57; G06T 2207/10016; G06T 2207/20081; G06T 2207/20084; G06T 2207/30201; G06T 2207/30232

(57) ABSTRACT

Systems and methods are provided for proctoring online exams that collects reference samples of a student's speech and video of them looking at their computer screen. Audio and video of the student are then captured during the exam. That audio and video is segmented and compared to the reference samples in order to identify segments that potentially contain at least one of another speaker who is not the student, the student looking away from the computer screen, another face that is not the student's. The frequency and severity of flagged segments are used to indicate the overall level of suspicion to an exam proctor.

### CROSS-REFERENCE TO RELATED APPLICATIONS

This application claims priority from U.S. provisional application 63/450,530 filed Mar. 7, 2023 the disclosure of which is incorporated herein by reference.

### FIELD OF THE DISCLOSURE

The present disclosure is generally related to online learning systems and the proctoring of online exams.

### DESCRIPTION OF THE RELATED ART

The instances of online learning have increased dramatically over the last decade. This increase in online courses has necessitated tens of thousands of online exams being administered.

Online and automated tools to prevent, reduce, and identify cheating are necessary to maintain the integrity of education delivered online. Current monitoring tools require dedicated browsers, devices, or large amounts of bandwidth to capture the necessary information.

Therefore it is desirable to have an automated proctoring solution to monitor multiple factors without requiring dedicated devices or high bandwidth connections.

### SUMMARY OF THE CLAIMED INVENTION

Embodiments of the present invention include systems and methods for proctoring online exams that collects reference samples of a student's speech and video of them looking at their computer screen. Audio and video of the student are then captured during the exam. That audio and video is segmented and compared to the reference samples in order to identify segments that potentially contain at least one of another speaker who is not the student, the student looking away from the computer screen, another face that is not the student's. The frequency and severity of flagged segments are used to indicate the overall level of suspicion to an exam proctor.

### BRIEF DESCRIPTIONS OF THE DRAWINGS

FIG. 1 illustrates a system for AI exam proctoring.

FIG. 2 Illustrates an example process of a proctoring base module.

FIG. 3 Illustrates an example process of a face matching module.

FIG. 4 Illustrates an example process of a gaze estimation module.

FIG. 5 Illustrates an example process of a speaker verification module.

FIG. 6 Illustrates an example process of a proctor verification module.

FIG. 7 illustrates an example of computing system.

FIG. 8 illustrates an example neural network architecture.

### DETAILED DESCRIPTION

Embodiments of the present disclosure will be described more fully hereinafter with reference to the accompanying drawings in which like numerals represent like elements throughout the several figures and in which example embodiments are shown. Embodiments of the claims may, however, be embodied in many different forms and should not be construed as limited to the embodiments set forth herein. The examples set forth herein are non-limiting examples and are merely examples among other possible examples.

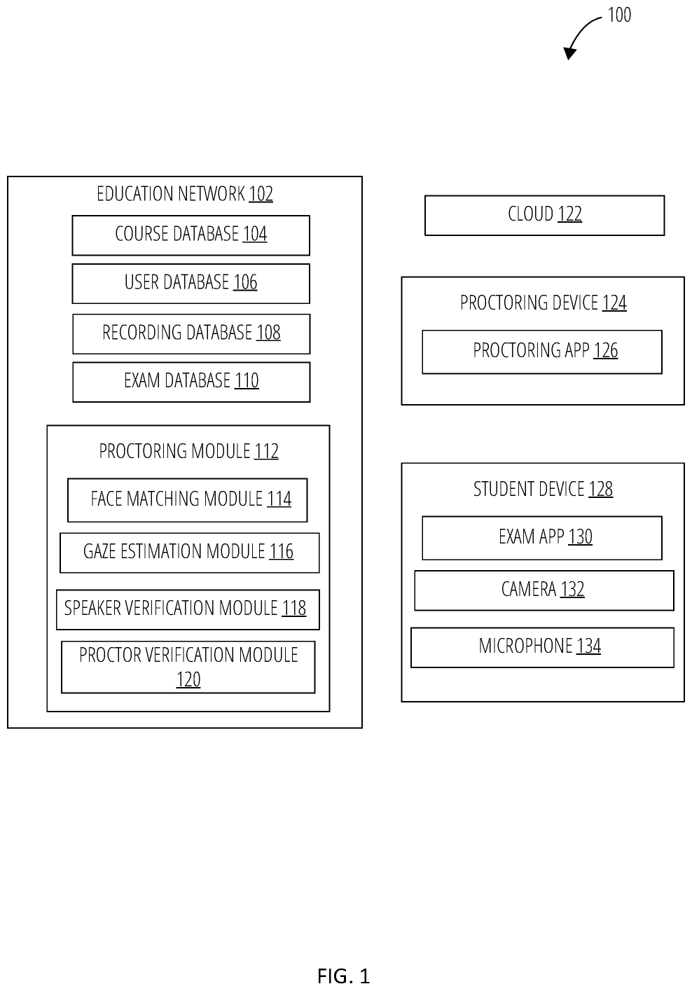

FIG. 1 is a system for AI exam proctoring.

This system comprises an education network 102, allowing users to take one or more courses online. The present invention is an artificial intelligence-driven system for proctoring online exams on the education network 102. The education network 102 may include a course database 104 that may store data related to courses available on the education network 102. Course information may include but is not limited to course titles, topics, syllabuses, resource materials, exams, quizzes, and instructors.

The education network 102 may include a user database 106 that may store data related to users of the education network 102. Users may include students, instructors, proctors, or administrators. User information may include contact information, device information, courses enrolled in, attendance, grades, etc.

The education network 102 may include a recording database 108 that may receive and store some or all of the video captured by the student device camera 132 and some or all of the audio captured by the student device microphone 134 while the student is taking a test through the exam app 130. In some cases, the audio and video are stored in the recording database 108 to be processed. In some cases, the data is segmented by an edge computing device such as the student device 128, and the segmented audio and video are stored in the recording database 108.

The education network 102 may include an exam database 110 that may store exam details such as questions, answers, resources, scores, etc. The education network 102 may include a proctoring base module 112 that may connect instructors, proctors, and students to the education network 102 to allow students to take exams while monitored for suspicious activity. The proctoring base module 112 may trigger the face matching module 114 to monitor any observed faces in the video from the student device camera 132 and compare observed faces to reference images of the user in order to identify situations in which there may be another person in the field of view who may be assisting the student, as well as identifying instances of attempting to spoof the camera 132.

The proctoring base module 112 may trigger the gaze estimation module 114 to monitor the pitch and yaw of the student's head to estimate if they are looking away from the computer screen, which may indicate suspicious activity such as looking at their phone or reference materials. The proctoring base module 112 may trigger the speaker verification module 118 to monitor the audio captured by the student device microphone 134 to identify voices that do not match the voice sample provided by the student, which may be indicative of the user receiving help from either another person or a digital assistant.

In some cases, the face matching module 114, the gaze estimation module 116, or the speaker verification module 118, may run on audio and video captured during the exam after the exam is complete and provide reports to the proctor. In some cases, the proctoring base module 112 may trigger the proctor verification module 120 to allow a proctor to monitor the students taking an exam in real-time, view flagged video or audio segments, and lockout students when necessary.

The education network 102 may include a cloud 122, or a communication network may be a wired and/or a wireless network to communicatively couple the education network 102 to proctor devices 124 and student devices 128. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network may allow ubiquitous access to shared pools of configurable system resources and higher-level services that can be rapidly provisioned with minimal management effort, often over the Internet, and relies on sharing of resources to achieve coherence and economies of scale like a public utility.

Furthermore, third-party clouds may enable organizations to focus on their core businesses instead of expending resources on computer infrastructure and maintenance. The education network 102 may include a proctor device 124 that may be a computing device such as a personal computer, laptop, smartphone, tablet, or smart speaker. The education network 102 may include a proctoring app 126 that may reside on the proctor device 124 and allow the proctor to access the education network 102 and receive proctor reports or proctor an exam in real-time through the proctor verification module 120.

The education network 102 may include a student device 128 that may be a computing device such as a personal computer, laptop, smartphone, tablet, or smart speaker. The education network 102 may include an exam app 130 that may reside on the student device 128 and allow students to access the education network 102 and take exams. Through the exam app 130, the student may provide sample images and audio of themselves to be used by the face matching module 114, the gaze estimation module 116, and the speaker verification module 118, as a reference to compare to continuous audio and video information from the student device camera 132 and the student device microphone 134 to identify potentially suspicious behavior by the student during the exam.

The education network 102 may include a camera 132 integrated with or communicatively coupled with the student device 128. The camera 132 captures video at 30 frames per second in one embodiment. The education network 102 may include a microphone integrated with or communicatively coupled with the student device 128.

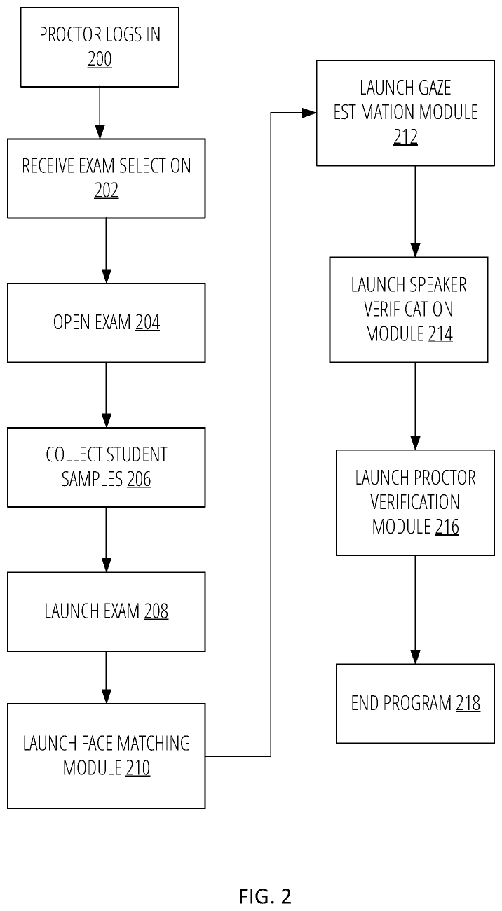

FIG. 2 illustrates an example process of the proctoring base module 112.

The process begins with a proctor logging into the system at step 200. The proctor may then select an exam from the exam database 110, and their selection is received at step 202. The selected exam may then be opened for students to take at step 204. There is a lobby for students to be in until the proctor opens the exam in one embodiment. Samples of the student's voice and video feed may be taken in the lobby. In the present example, the samples from the student device microphone 134 and student device camera 132 may be taken at step 206. Once audio and video samples have been collected from all students, the exam is launched at step 208.

Launching the exam allows students to begin answering questions. Once the exam is launched, the face matching module 114 is launched at step 210. The face matching module 114 will examine segments of the video feed from the student device camera 132 and compare the identified faces to the reference sample provided by the student to identify suspicious situations such as another student helping the student or the student trying to spoof the camera. Then the gaze estimation module 116 is then launched at step 212. The gaze estimation module 116 will monitor the pitch and yaw of the student's head to identify when the student may be looking away from the screen, which may indicate cheating. Then the speaker verification module 118 is launched at step 214. The speaker verification module 118 will compare audio segments received from the student device microphone 134 to identify voices that do not match the student's sample to identify voices that do not match as potentially suspicious. In embodiments in which the proctor will be monitoring the exam live, the proctor verification module 120 is launched at step 216. The four launched modules will continue to run until the exam is complete. The program ends at step 218.

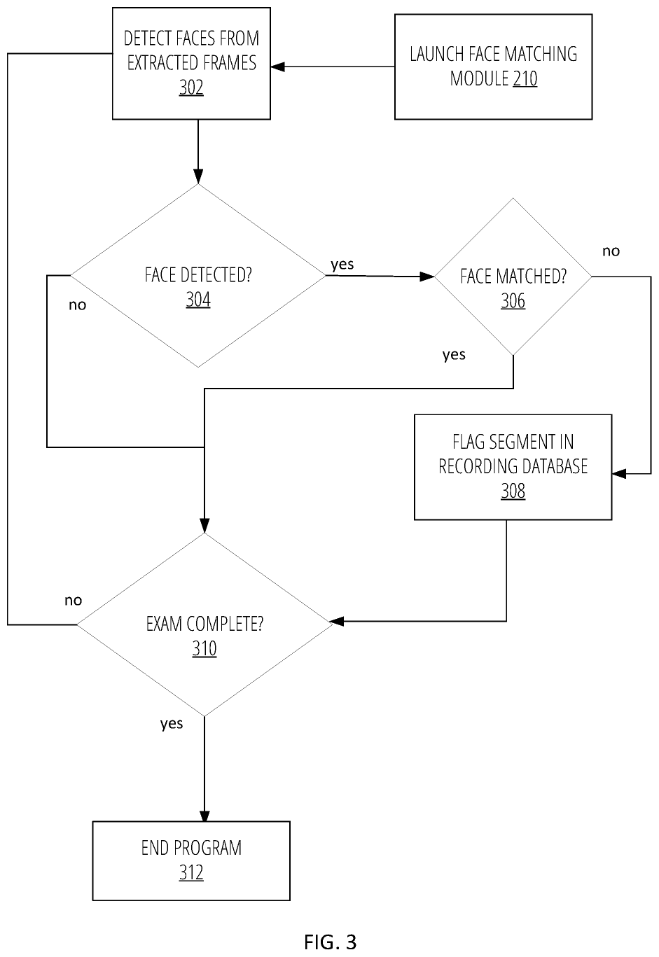

FIG. 3 illustrates an example process of the face matching module 114.

The process begins with launching the face matching module 114 at step 210. Once launched, the video may be received from the student device camera 132. A frame extraction service may extract video frames from a video in regularly spaced time intervals. In some cases, a fixed number of frames are extracted at every time interval, and all the extracted frames are batched into separate chunks of NumPy arrays. The chunks may then be saved to the recording database 108. A frame extraction rate may control the time interval at which frames are extracted regularly from the video, and in some cases, ‘15 s’ may be an appropriate value. With a larger time interval, the model may miss out on important details in the video, whereas a smaller interval does not provide much gain in information.

The number of frames per timestamp may be a parameter that controls the number of frames to be extracted per time interval. More specifically, first ‘n’ frames are extracted at each time interval, and the value of ‘n’ is controlled by this parameter. In some cases, a default value of 10 may be chosen. This chosen value may be susceptible to the recorded video's frames per second (fps). By default, video may be captured at 30 fps, so 10 frames may represent a time interval of ⅓ of a second.

With a smaller value, the model tends to miss out on blurry faces, which can occur from sudden movement of the examinee, and with much larger values, the model may not be able to detect the examinee when they are missing from the frame. Another problem with using a large value is increased computation cost. The value of 10 frames per time interval for a 30 fps video provides a good tradeoff between the two extremes.

A face detection service may be applied to the extracted frames at step 302. Face detection service loads the extracted frames from the recording database 108 and determines, at step 304, if there are detected faces from those extracted frames. The Face detection service may detect cases where no faces are present, a single face is present, and multiple faces are present. The face detection service may return a cropped and aligned image of just the face that is then resized to match the input size for the face recognition model employed by a face matching service. The detected faces in the video may be saved as pickle files in the recording database 108.

If there are faces detected, the detected face in the student-provided sample is then used to compare the detected faces in the video by face-matching service. It is saved as a NumPy array and dumped into recording database 108. Face matching may be done by calculating the Euclidean distance between the embeddings of every face detected in video frames and the face detected from the sample image. It may then be determined if the detected face matches the sample face at step 306. If the Euclidean distance exceeds a certain threshold, that particular timestamp on the video is flagged in the recording database at step 308. With the conditions: no face and multiple faces detected by the face detection service and the condition of face mismatch detected here, a final response may be generated, which is a JSON object that has the following fields:

No face: This field contains a list of timestamps where the face detection model detected no face.

Multiple faces: This field contains a list of timestamps where the face detection model detected more than one face.

Mismatched faces: This field contains a list of timestamps where the face matching service could not match the face in the video with the face in the profile picture.

Each video segment may also be examined for face spoofing. It may then be determined if the exam is complete at step 310. If the exam is not complete, the process returns to step 302. If the exam is complete, the program ends at step 312.

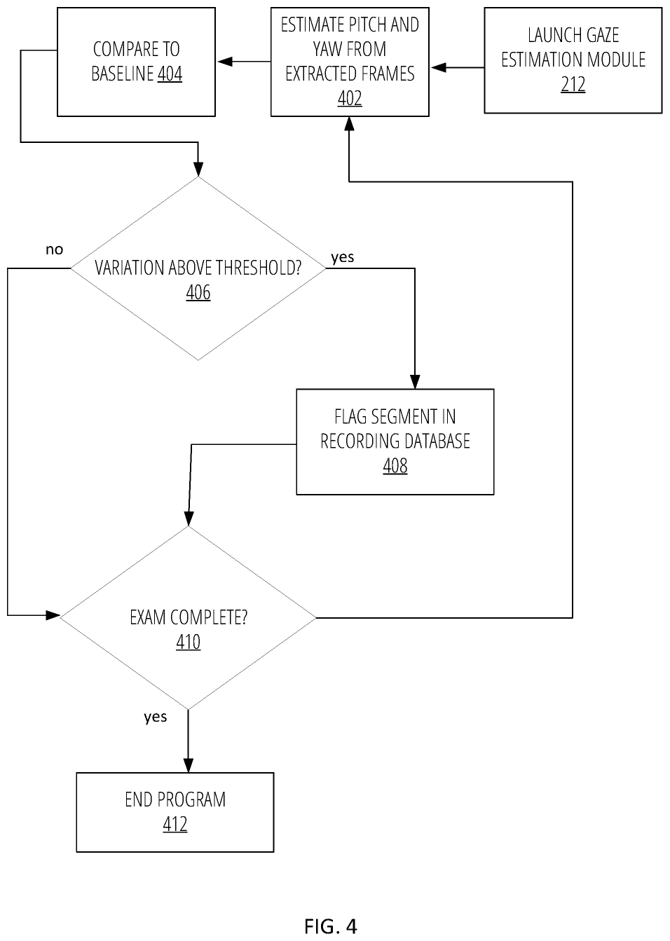

FIG. 4 illustrates an example process of the gaze estimation module 116. The process begins launching the proctoring base module 112 at step 212. Once launched, a video may be received from the student device camera 132. A frame extraction service may extract video frames from a video in regularly spaced time intervals. A fixed number of frames are extracted at every time interval, and all the extracted frames are batched into separate chunks of NumPy arrays. The chunks are then saved to recording database 108.

Gaze estimation may be the process of estimating the student's gaze to monitor the student's behavior during a proctored exam. In some cases, this is done by monitoring the yaw angle, i.e., the student looking left or right of the screen, or the pitch angle, i.e., the student looking up or down of the screen being monitored. The yaw and pitch of the student's head are estimated at step 402. The estimated yaw and pitch are then compared to the baseline yaw and pitch from the sample video at step 404.

In some cases, the reference video is captured while the student is taking the exam, in some cases it may be a first period of time such as couple of seconds or minutes of the student taking the exam. In some cases, the reference video does not need to show the user demonstrating the extent of the yaw angle and the pitch angle but may be calculated by a machine-learning model of the gaze estimation module. The machine-learning model may determine the baseline yaw and pitch based on training data that associates head and neck shapes with respective yaw and pitch angles. In some cases, a standard baseline yaw and pitch may be updated as the reference video accumulates more datapoints.

A reference video may be used as a baseline because different users will have different head angles, and they will be impacted by the position and angle of the camera 132. To calculate the normal yaw and pitch angle, 10 frames of the user may be taken before the proctored exam and calculate the normal yaw and pitch angle. This baseline pitch and yaw of the student's head while viewing the computer screen can be used as a threshold. At every 15s, 10 frames may be captured to calculate yaw and pitch angle and may be compared with a similarity threshold, i.e. [normal yaw and pitch angle+−20]. In some cases, plus, or minus twenty degrees may be used. It may then be determined if the current yaw or pitch angle exceeds the threshold at step 406. If a certain percentage such as 70%, of the frames at every timestamp are greater or less than the threshold, the segment may be flagged in the recording database 108, at step 408. It may then be determined if the exam is complete at step 410. If the exam is not complete, the process returns to step 402. If the exam is complete, the program ends at step 412.

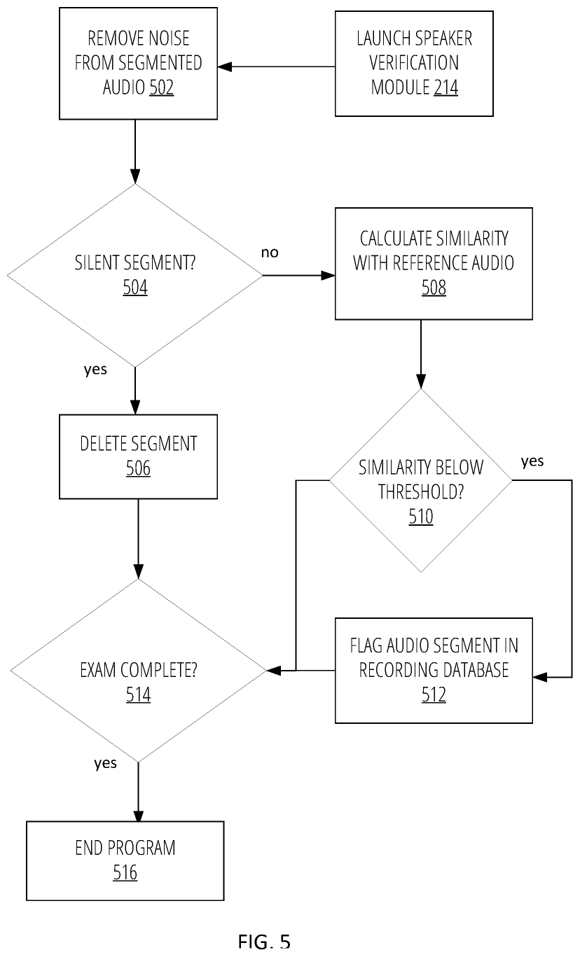

FIG. 5 illustrates an example process of the speaker verification module 118. The process begins with launching the speaker verification module 118 at step 214. Once launched, the audio may be received from the student device microphone 134, and audio may be segmented. In some cases, the segments are four-second intervals. Background noise may then be removed from audio segments in step 502. Background noise may be removed in many ways. In some cases, noisy segments are removed entirely. A noise reduction filter may be applied to the audio segments to remove background noise in an ideal embodiment. After filtering background noise, it may be determined if the audio segment is silent at step 504. If the segment is silent, the segment is deleted at step 506. Silent segments are removed as they do not need to be processed. If the segment is not silent, the similarity between the audio segment and the reference audio captured from the student is calculated at step 508. Speaker verification may be done by calculating the cosine similarity between segment and reference audio.

Text-independent speaker verification (TI-SV) may not have lexicon constraints for the system to use as a reference, which may result in large variability of phonemes and utterance durations. Generalized end-to-end (GE2E) training of neural networks to recognize speech may be done by processing many utterances at once. Processing many utterances at once, typically in batches, allows the network to learn more robust and generalized representations. This is because the network can observe a variety of speech patterns and speaker characteristics within each batch, leading to more effective learning. Batches may contain N speakers and M utterances from each speaker, with each featuring a vector xji (1≤j≤N and 1≤i≤M), represents the features extracted from speaker j utterance i.

A similarity matrix may be defined as the scaled cosine similarities between each embedding vector. In some cases, the same speaker speaking in the same language will have 86-88% similarity on average. The same speaker speaking in a different language will have, on average, 82% similarity. A different speaker speaking the same language will have an average similarity score of 54%, and a different speaker speaking a different language will also have a similarity score averaging 54%. In some cases, a similarity threshold of 67% is defined. It may be determined if the audio segment contains an utterance below the similarity threshold at step 514. If the audio segment contains an utterance below the similarity threshold, the segment is flagged as suspicious in the recording database 108, at step 516. It may then be determined if the exam is complete at step 518. If the exam is not complete, the process returns to step 502. If the exam is complete, the program ends at step 520.

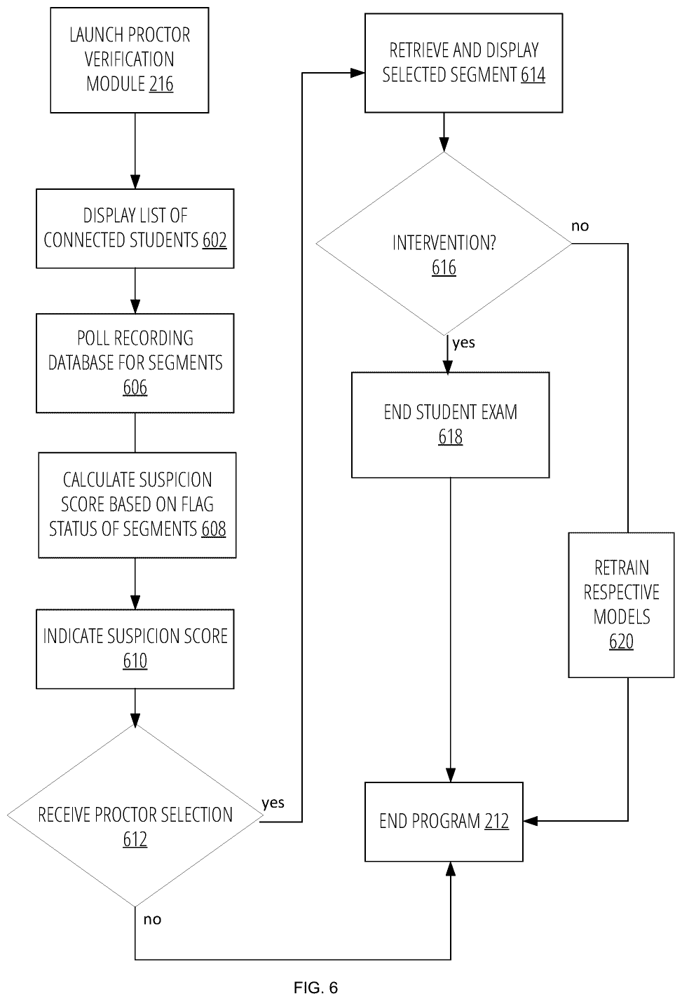

FIG. 6 illustrates an example process of the proctor verification module 120. The process begins with receiving a prompt from the proctoring base module 112 at step 600. All students currently taking the exam may be displayed on the proctor device 124, at step 602. The names associated with the students may be displayed in a list. In one embodiment, the video feed from the student device cameras 132 may be displayed, or still, images of each student may be displayed.

The recording database 108 may be polled for new video or audio segments that have been processed by either the face matching module 114, the gaze estimation module 116, or the speaker verification module 118, at step 604. When new segments are received, identify, and display the flag on the proctor device 124, at step 606. For example, the proctor device 124 may display a series of blocks under the student's name, with each block indicating a portion of time during the exam.

For example, the blocks may be colored green when the segment is not suspicious and red when at least one module has flagged that segment in the recording database 108. There may be an intermediate flag, for example, yellow, for segments near a threshold, such as a voice match that is 65% similar would be 1% above the threshold for being flagged as suspicious. The suspicion score may be calculated at step 608. The suspicion score may indicate the suspicion level of the student's activity during the exam. This indication could be a simple yes or no indication of the presence of any audio or video segments that have been flagged by any one of the face matching module 114, the gaze estimation module 116, or the speaker verification module 118.

There may be a threshold number of segments, such as 10 segments, before a suspicion threshold is met in one embodiment. Students above or below this threshold of suspicious segments may have a red or green indicator of their suspicion score displayed for the proctor. The suspicion score may be based on the percentage of flagged segments. For example, students with less than 0.1% of segments flagged may have a suspicion score of 0, and those with at least 5% of segments flagged may have a suspicion score of 100, while suspicion scores may scale linearly between those values. The suspicion score may be indicated to the proctor at step 610. It may then be determined if the proctor selects a student or segment at step 612, for example, in the embodiment in which segments are represented by blocks that are color-coded (red, yellow, green) for their level of suspicion.

A proctor may select a given segment to review. When the proctor selects a segment, it is retrieved from the recording database 108, at step 614. The retrieved segment may then be displayed on the proctor device 124, at step 616. It may be determined if the proctor wants to intervene in the student's exam based on viewing the retrieved segment or seeing their suspicion score at step 618. If the proctor elects to intervene in the student's exam, the student's exam is ended at step 620. For example, the proctor may select an audio segment with a similarity score below the 64% threshold from a student. The segment is retrieved and played on the proctor device 124. The proctor may agree with the software that two distinct speakers are heard in the audio segment and elect to terminate the student's exam. In one embodiment, the proctor may view or hear the segments before or after the flagged segment or may be able to view the entirety of the audio/video data. It may then be determined if the exam is complete at step 622. If the exam is not complete, the process returns to step 604. If the exam is complete, the program ends at step 624.

In some cases, the indicated suspicion score may inaccurately indicate that there is any issue with the flagged segment. When the proctor decides not to intervene, respective models may be retrained to adjust the weights to deprioritize aspects associated with the flagged segments. In some cases, the proctor is presented a display that generates real-time indications of the suspicion scores and may be given an indication to review a change to the standard baseline yaw and pitch by showing a respective flagged segment.

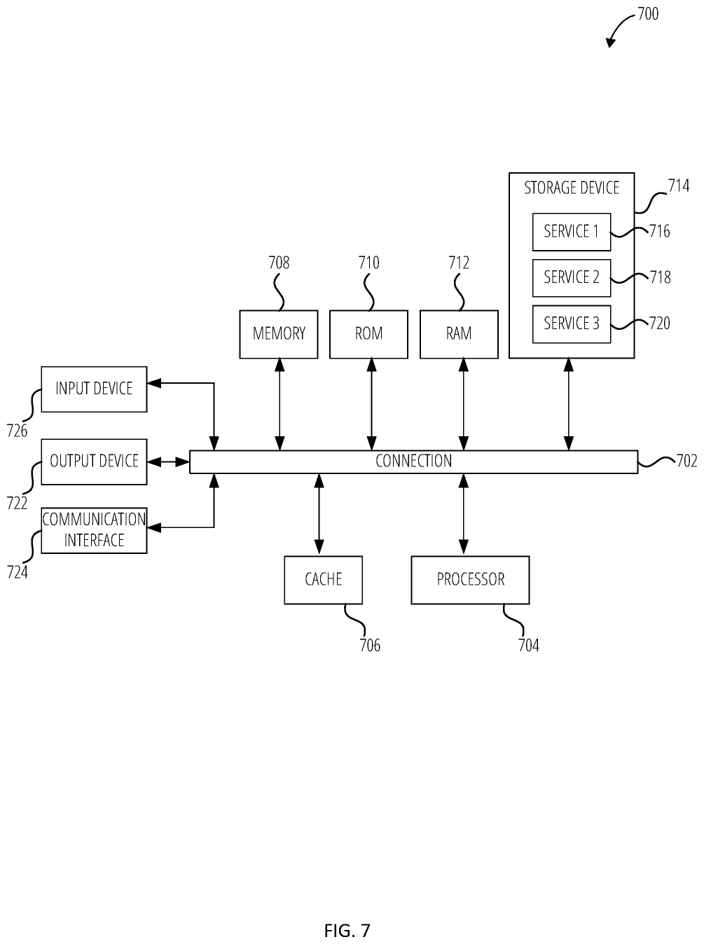

FIG. 7 shows an example of computing system 700, which can be for example any computing device making up education network 102, or any component thereof in which the components of the system are in communication with each other using connection 702. Connection 702 can be a physical connection via a bus, or a direct connection into processor 704, such as in a chipset architecture. Connection 702 can also be a virtual connection, networked connection, or logical connection.

In some embodiments, computing system 700 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing system 700 includes at least one processing unit (CPU or processor) 704 and connection 702 that couples various system components including system memory 708, such as read-only memory (ROM) 710 and random access memory (RAM) 712 to processor 704. Computing system 700 can include a cache of high-speed memory 708 connected directly with, in close proximity to, or integrated as part of processor 704.

Processor 704 can include any general purpose processor and a hardware service or software service, such as services 706, 718, and 720 stored in storage device 714, configured to control processor 704 as well as a special-purpose processor where software instructions are incorporated into the actual processor design. Processor 704 may essentially be a completely self-contained computing system, containing multiple cores or processors, a bus, memory controller, cache, etc. A multi-core processor may be symmetric or asymmetric.

To enable user interaction, computing system 700 includes an input device 726, which can represent any number of input mechanisms, such as a microphone for speech, a touch-sensitive screen for gesture or graphical input, keyboard, mouse, motion input, speech, etc. Computing system 700 can also include output device 722, which can be one or more of a number of output mechanisms known to those of skill in the art. In some instances, multimodal systems can enable a user to provide multiple types of input/output to communicate with computing system 700. Computing system 700 can include communication interface 724, which can generally govern and manage the user input and system output. There is no restriction on operating on any particular hardware arrangement, and therefore the basic features here may easily be substituted for improved hardware or firmware arrangements as they are developed.

Storage device 714 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 714 can include software services, servers, services, etc., that when the code that defines such software is executed by the processor 704, it causes the system to perform a function. In some embodiments, a hardware service that performs a particular function can include the software component stored in a computer-readable medium in connection with the hardware components, such as processor 704, connection 702, output device 722, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

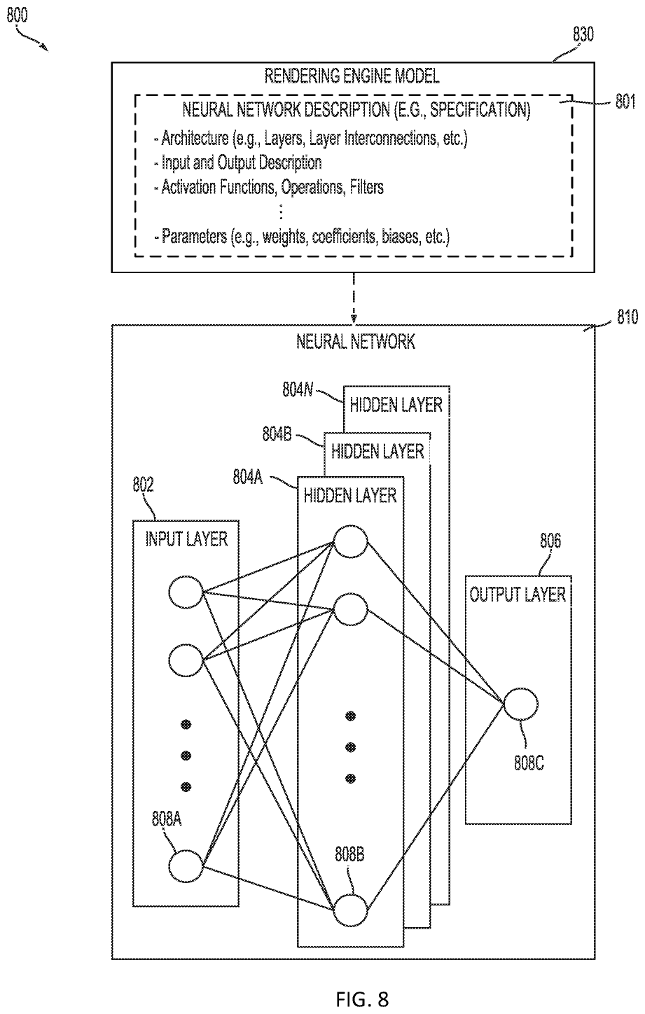

FIG. 8 illustrates an example neural network architecture. Architecture 800 includes a neural network 810 defined by an example neural network description 801 in rendering engine model (neural controller) 830. The neural network 810 can represent a neural network implementation of a rendering engine for rendering media data. The neural network description 801 can include a full specification of the neural network 810, including the neural network architecture 800. For example, the neural network description 801 can include a description or specification of the architecture 800 of the neural network 810 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 810 reflects the architecture 800 defined in the neural network description 801. In this example, the neural network 810 includes an input layer 802, which includes input data, such as images/videos or audio. In one illustrative example, the input layer 802 can include data representing a portion of the input media data such as a patch of data or pixels (e.g., a 128×128 patch of data) in an image corresponding to the input media data (e.g., images/videos or audio).

The neural network 810 includes hidden layers 804A through 804 N (collectively “804” hereinafter). The hidden layers 804 can include n number of hidden layers, where n is an integer greater than or equal to one. The number of hidden layers can include as many layers as needed for a desired processing outcome and/or rendering intent. The neural network 810 further includes an output layer 806 that provides an output (e.g., flagging of images/videos or audio) resulting from the processing performed by the hidden layers 804. In one illustrative example, the output layer 806 can provide flagging of images/videos or audio.

The neural network 810 in this example is a multi-layer neural network of interconnected nodes. Each node can represent a piece of information. Information associated with the nodes is shared among the different layers and each layer retains information as information is processed. In some cases, the neural network 810 can include a feed-forward neural network, in which case there are no feedback connections where outputs of the neural network are fed back into itself. In other cases, the neural network 810 can include a recurrent neural network, which can have loops that allow information to be carried across nodes while reading in input.

Information can be exchanged between nodes through node-to-node interconnections between the various layers. Nodes of the input layer 802 can activate a set of nodes in the first hidden layer 804A. For example, as shown, each of the input nodes of the input layer 802 is connected to each of the nodes of the first hidden layer 804A. The nodes of the hidden layer 804A can transform the information of each input node by applying activation functions to the information. The information derived from the transformation can then be passed to and can activate the nodes of the next hidden layer (e.g., 804B), which can perform their own designated functions. Example functions include convolutional, up-sampling, data transformation, pooling, and/or any other suitable functions. The output of the hidden layer (e.g., 804B) can then activate nodes of the next hidden layer (e.g., 804 N), and so on. The output of the last hidden layer can activate one or more nodes of the output layer 806, at which point an output is provided. In some cases, while nodes (e.g., nodes 808A, 808B, 808C) in the neural network 810 are shown as having multiple output lines, a node has a single output and all lines shown as being output from a node represent the same output value.

In some cases, each node or interconnection between nodes can have a weight that is a set of parameters derived from training the neural network 810. For example, an interconnection between nodes can represent a piece of information learned about the interconnected nodes. The interconnection can have a numeric weight that can be tuned (e.g., based on a training dataset), allowing the neural network 810 to be adaptive to inputs and able to learn as more data is processed.

The neural network 810 can be pre-trained to process the features from the data in the input layer 802 using the different hidden layers 804 in order to provide the output through the output layer 806. In an example in which the neural network 810 is used to flag certain images/videos or audio, the neural network 810 can be trained using training data that includes example images and facial features of user 802 and/or labeling and characteristic information (e.g., name, brand, size, etc.) of product(s) 808. For instance, training images can be input into the neural network 810, which can be processed by the neural network 810 to generate outputs which can be used to tune one or more aspects of the neural network 810, such as weights, biases, etc.

In some cases, the neural network 810 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For a first training iteration for the neural network 810, the output can include values that do not give preference to any particular class due to the weights being randomly selected at initialization. For example, if the output is a vector with probabilities that the object includes different product(s) and/or different users, the probability value for each of the different product and/or user may be equal or at least very similar (e.g., for ten possible products or users, each class may have a probability value of 0.1). With the initial weights, the neural network 810 is unable to determine low level features and thus cannot make an accurate determination of what the classification of the object might be. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. The neural network 810 can perform a backward pass by determining which inputs (weights) most contributed to the loss of the neural network 810, and can adjust the weights so that the loss decreases and is eventually minimized.

A derivative of the loss with respect to the weights can be computed to determine the weights that contributed most to the loss of the neural network 810. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 810 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, the neural network 810 can represent any other neural or deep learning network, such as an autoencoder, a deep belief nets (DBNs), a recurrent neural networks (RNNs), etc.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

What is claimed is:

1. A system for proctoring online exams, the system comprising:
memory that stores a face detection model, a gaze estimation model, a speaker verification module and a proctor verification module;
a communication interface that communicates over a communication network with one or more student devices, wherein the communication interface initiates, over an education network hosting online exams, an online exam for one or more students at the respective one or more student devices connected to the education network; and
a processor that executes instructions stored in the memory, wherein the processor executes the instructions to:
extract video frames from a video recorded from a camera of one of the student devices;
apply the face detection model to the extracted frames, wherein the face detection model compares detected faces to a provided image, wherein when no face is detected, more than one face is detected, or a face mismatch is detected, respective frames are flagged in a recording database;
apply the gaze estimation model to the extracted frames by monitoring a yaw and pitch of a detected head in the extracted frames and comparing the yaw and pitch with a baseline yaw and pitch of the respective student, wherein video segments with frames that have a yaw and pitch below a first similarity threshold in comparison to the baseline yaw and pitch are flagged;
apply the speaker verification model to audio segments to remove silent segments and flagging audio segments with an utterance below a second similarity threshold based on a comparison with a reference audio captured from the student; and
apply the proctor verification module to calculate suspicion scores of flagged segments and determining that an intervention is required.

2. The system of claim 1, wherein the face detection model compares the detected faces to the provided image by calculating a Euclidean distance between an embedding of each face detected in the extracted frames and a face detected from the provided image.

3. The system of claim 1, wherein the audio segments are flagged by using trained neural networks that recognize speech by processing many utterances at once, wherein vectors featuring a plurality of speakers and a plurality of utterance from each speaker are batched.

4. The system of claim 3, wherein a similarity matrix defined as scaled cosine similarities extracted from one speaker and one utterance, and wherein the second similarity threshold is determined based on the similarity of compared vectors.

5. The system of claim 1, wherein the processor further executes the instructions to:
receive an indication to not intervene based on the flagged segments; and
retrain the face detection model, the gaze estimation model, or the speaker verification model based on the respective model that flagged the flagged segments, wherein the retraining adjusts weights to deprioritize aspects associated with the flagged segments.

6. The system of claim 1, wherein the baseline yaw and pitch are calculated from a reference video, and wherein a yaw angle is associated with the respective student looking left or right of a screen and a pitch angle is associated with the respective student looking up and down from the screen.

7. The system of claim 1, wherein the baseline yaw and pitch are calculated from a reference video of a time period of the respective student taking the exam, wherein a standard baseline yaw and pitch is updated as a machine-learning model accumulates more datapoints from the reference video in real time.

8. The system of claim 7, wherein the processor further executes the instructions to:
cause to present a display that generates real-time indications of the suspicion scores; and
receive an indication to review a change to the standard baseline yaw and pitch by showing a respective flagged segment.

9. A method of proctoring online exams, the method comprising:
initiating, over an education network hosting online exams, an online exam for one or more students at respective one or more student devices connected to the education network;
extracting video frames from a video recorded from a camera of one of the student devices;
applying a face detection model to the extracted frames, wherein the face detection model compares detected faces to a provided image, wherein when no face is detected, more than one face is detected, or a face mismatch is detected, respective frames are flagged in a recording database;
applying a gaze estimation model to the extracted frames by monitoring a yaw and pitch of a detected head in the extracted frames and comparing the yaw and pitch with a baseline yaw and pitch of the student, wherein video segments with frames that have a yaw and pitch below a first similarity threshold in comparison to the baseline yaw and pitch are flagged;
applying a speaker verification model to audio segments to remove silent segments and flagging audio segments with a utterance below a second similarity threshold based on a comparison with a reference audio captured from the student; and
applying a proctor verification module to calculate suspicion scores of flagged segments and determining that an intervention is required.

10. The method of claim 9, wherein the face detection model compares the detected faces to the provided image by calculating a Euclidean distance between an embedding of each face detected in the extracted frames and a face detected from the provided image.

11. The method of claim 9, wherein the audio segments are flagged by using trained neural networks that recognize speech by processing many utterances at once, wherein vectors featuring a plurality of speakers and a plurality of utterance from each speaker are batched.

12. The method of claim 11, wherein a similarity matrix defined as scaled cosine similarities extracted from one speaker and one utterance, and wherein the second similarity threshold is determined based on the similarity of compared vectors.

13. The method of claim 9, further comprising:
receiving an indication to not intervene based on the flagged segments; and
retraining the face detection model, the gaze estimation model, or the speaker verification model based on the respective model that flagged the flagged segments, wherein the retraining adjusts weights to deprioritize aspects associated with the flagged segments.

14. The method of claim 9, wherein the baseline yaw and pitch are calculated from a reference video, and wherein a yaw angle is associated with the student looking left or right of a screen and a pitch angle is associated with the student looking up and down from the screen.

15. The method of claim 9, wherein the baseline yaw and pitch are calculated from a reference video of a time period of the student taking the exam, wherein a standard baseline yaw and pitch is updated as a machine-learning model accumulates more datapoints from the reference video in real time.

16. The method of claim 15, further comprising:
causing to present a display that generates real-time indications of the suspicion scores; and
receiving an indication to review a change to the standard baseline yaw and pitch by showing a respective flagged segment.

17. A non-transitory, computer-readable storage medium, having embodied thereon instructions executable by a computing system to perform a method for proctoring online exam, the method comprising:
initiating, over an education network hosting online exams, an online exam for one or more students at respective one or more student devices connected to the education network;
extracting video frames from a video recorded from a camera of one of the student devices;
applying a face detection model to the extracted frames, wherein the face detection model compares detected faces to a provided image, wherein when no face is detected, more than one face is detected, or a face mismatch is detected, respective frames are flagged in a recording database;
applying a gaze estimation model to the extracted frames by monitoring a yaw and pitch of a detected head in the extracted frames and comparing the yaw and pitch with a baseline yaw and pitch of the student, wherein video segments with frames that have a yaw and pitch below a first similarity threshold in comparison to the baseline yaw and pitch are flagged;
applying a speaker verification model to audio segments to remove silent segments and flagging audio segments with a utterance below a second similarity threshold based on a comparison with a reference audio captured from the student; and
applying a proctor verification module to calculate suspicion scores of flagged segments and determining that an intervention is required.

18. The non-transitory, computer-readable storage medium of claim 17, wherein the face detection model compares the detected faces to the provided image by calculating an Euclidean distance between an embedding of each face detected in the extracted frames and a face detected from the provided image.

19. The non-transitory, computer-readable storage medium of claim 17, wherein the audio segments are flagged by using trained neural networks that recognize speech by processing many utterances at once, wherein vectors featuring a plurality of speakers and a plurality of utterance from each speaker are batched.

20. The non-transitory, computer-readable storage medium of claim 19, wherein a similarity matrix defined as scaled cosine similarities extracted from one speaker and one utterance, and wherein the second similarity threshold is determined based on the similarity of compared vectors.
